(19) United States
(12) Patent Application Publication
(10) Pub. No.: US 2003/0035773 A1
(43) Pub. Date: Feb. 20, 2003

(54) System and method for quantitative assessment of joint diseases and the change over time of joint diseases

(75) Inventors: Saara Marjatta Sofia Totterman, Rochester, NY (US); Jose Tamez-Pena, Rochester, NY (US); Edward Ashton, Webster, NY (US); Kevin J. Parker, Rochester, NY (US)

Correspondence Address: BLANK ROME COMISKY & MCCAULEY, LLP, 900 17TH STREET, N.W., SUITE 1000, WASHINGTON, DC, 20006, US

(73) Assignee: VirtualScopics LLC

(21) Appl. No.: 10/205,606

(22) Filed: Jul. 26, 2002

Related U.S. Application Data
(60) Provisional application No. 60/307,869, filed on Jul. 27, 2001.

(51) Int. Cl. A61K 49/00; G06F 19/00; G01N 33/48; G01N 33/50; G06K 9/00; A61B 5/00

(52) U.S. Cl. 424/9.1; 600/300; 382/128; 702/19

(57) ABSTRACT

In a human or animal joint, specific objects serve as indicators, or biomarkers, of joint disease. In a three-dimensional image of the joint, the biomarkers are identified and quantified. Multiple three-dimensional images can be taken over time, in which the biomarkers can be tracked over time. Statistical segmentation techniques are used to identify the biomarker in a first image and to carry the identification over to the remaining images.

### REFERENCE TO RELATED APPLICATIONS

The present application claims the benefit of U.S. Provisional Application No. 60/307,869, filed Jul. 27, 2001, whose disclosure is hereby incorporated by reference in its entirety into the present disclosure.

### FIELD OF THE INVENTION

The present invention is directed to a system and method for quantitative assessment of joint diseases and their change over time and is more particularly directed to such a system and method which use biomarkers.

### DESCRIPTION OF RELATED ART

Diseases of the joints, such as osteoarthritis and other degenerative and post-traumatic diseases, afflict a significant percent of the population. In addition, there are a number of injuries to the knee, shoulder, elbow, wrist, ankle, and other complex joints and their supporting ligaments and structures, that unfortunately lead to a progression of diminished function. In assessing those conditions, and in tracking their change over time, including improvements due to new therapies, it is necessary to have quantitative information. Subjective measures of pain or discomfort have been used in the past. Less subjective measures can be obtained from measurements of images on x-ray films and digital x-ray images, but those are traditionally assessed by manual tracings or caliper measurements of the image. With the availability of 3D image sets from MRI and CT scanners, more detailed manual assessments can be obtained, usually by tracing of an object of interest using a mouse or trackball interfaced to the image workstation. Examples of measurements that are taken in osteoarthritis of both human and animal knee include: the thickness of the cartilage, the volume of the cartilage, the image intensity of the cartilage and bone, and the T2 relaxation time of the cartilage.

Some references for the prior work include: Eckenstein F., Gavazzeni H. S., Sittek H., Haubner, M., Losch, A., Milz, S., Englmeier, K-H., Schulte, E., Putz, R, Reiser, M., “Determination of Knee Joint Cartilage Thickness using Three-Dimensional Magnetic Resonance Chondro-Crassometry (3D MR-CCM),” Magnetic Resonance in Medicine 36:256-265, 1996; Solloway, S., Hutchinson, C. E., Waterton, J. C., Taylor, C., “The Use of Active Shape Models for Making Thickness Measurements of Articular Cartilage from MR Images,” Magnetic Resonance in Medicine 37:943-952, 1997; Stammberger, T., Eckstein, F., Englmeier, K-H., Reiser, M., “Determination of 3D Cartilage Thickness Data from MR Imaging: Computational Method and Reproducibility in the Living,” Magnetic Resonance in Medicine 41: 529-536, 1999; Ghosh, S., Ries, M., Lane, N., Majundar, S. “Segmentation of High Resolution Articular Cartilage MR Images,” 46th Annual Meeting, Orthopaedic Research Society, Mar. 12-15, 2000, Orlando Fla.; Dardzinski, B. J., Mosher, T. J., Li, S., Van Slyke, M. A., Smith, M. B., “Spatial Variation of T2 in Human Articular Cartilage, Radiology 205: 546-550, 1997. Those measurements require manual or semi-manual systems that require a user to identify the structure of interest and to trace boundaries or areas, or to initialize an active contour.

The prior art is capable of assessing gross abnormalities or gross changes over time. However, the conventional measurements are not well suited to assessing and quantifying subtle abnormalities, or subtle changes, and are incapable of describing complex topology or shape in an accurate manner. Furthermore, manual and semi-manual measurements from raw images suffer from a high inter-space and intra-observer variability. Also, manual and semi-manual measurements tend to produce ragged and irregular boundaries in 3D when the tracings are based on a sequence of 2D images.

### SUMMARY OF THE INVENTION

It will be readily apparent that a need exists in the art to overcome the above-noted difficulties associated with manual and semi-manual measurements from raw images and with the use of 2D images.

It is therefore a primary object of the invention to provide a more accurate quantification of joints and their diseases. It is another object of the invention to provide a more accurate quantification of changes in time of joint diseases. It is a further object of the invention to address the needs noted above.

To achieve the above and other objects, the present invention is directed to the identification of important structures or substructures, their normalities and abnormalities, and the identification of their specific topological, morphological, radiological, and pharmacokinetic characteristics which are sensitive indicators of joint disease and the state of pathology. The abnormality and normality of structures, along with their topological and morphological characteristics and radiological and pharmacokinetic parameters, are called biomarkers, and specific measurements of the biomarkers serve as the quantitative assessment of joint disease.

The inventors have discovered that the following new biomarkers are sensitive indicators of osteoarthritis joint disease in humans and in animals:

shape of the subchondral bone plate

layers of the cartilage and their relative size

signal intensity distribution within the cartilage layers

contact area between the articulating cartilage surfaces

surface topology of the cartilage shape

intensity of bone marrow edema

separation distances between bones

meniscus shape

meniscus surface area

meniscus contact area with cartilage

cartilage structural characteristics

cartilage surface characteristics

meniscus structural characteristics

meniscus surface characteristics

pannus structural characteristics

joint fluid characteristics

osteophyte characteristics

bone characteristics

lytic lesion characteristics

prosthesis contact characteristics

prosthesis wear

joint spacing characteristics

tibia medial cartilage volume

Tibia lateral cartilage volume

femur cartilage volume

patella cartilage volume

tibia medial cartilage curvature

tibia lateral cartilage curvature

femur cartilage curvature

patella cartilage curvature

cartilage bending energy

subchondral bone plate curvature

subchondral bone plate bending energy

meniscus volume

osteophyte volume

cartilage T2 lesion volumes

bone marrow edema volume and number

synovial fluid volume

synovial thickening

subchondrial bone cyst volume

kinematic tibial translation

kinematic tibial rotation

kinematic tibial valcus

distance between vertebral bodies

degree of subsidence of cage

degree of lordosis by angle measurement

degree of off-set between vertebral bodies

femoral bone characteristics

patella characteristics.

The preferred technique for extracting the biomarkers is with statistical based reasoning as defined in Parker et al (U.S. Pat. No. 6,169,817), whose disclosure is hereby incorporated by reference in its entirety into the present disclosure. The preferred method for quantifying shape and topology is with the morphological and topological formulas as defined by the following references:

Curvature Analysis: Peet, F. G., Sahota, T. S., “Surface Curvature as a Measure of Image Texture” IEEE Transactions on Pattern Analysis and Machine Intelligence 1985 Vol PAMI-7 G:734-738.

Struik, D. J., Lectures on Classical Differential Geometry, 2nd ed., Dover, 1988.

Shape and Topological Descriptors: Duda, R. O, Hart, P. E., Pattern Classification and Scene Analysis, Wiley & Sons, 1973.

Jain, A. K, Fundamentals of Digital Image Processing, Prentice Hall, 1989.

Spherical Harmonics: Matheny, A., Goldgof, D., “The Use of Three and Four Dimensional Surface Harmonics for Nonrigid Shape Recovery and Representation,” IEEE Transactions on Pattern Analysis and Machine Intelligence 1995, 17: 967-981; Chen, C. W, Huang, T. S., Arrot, M., “Modeling, Analysis, and Visualization of Left Ventricle Shape and Motion by Hierarchical Decomposition,” IEEE Transactions on Pattern Analysis and Machine Intelligence 1994, 342-356.

Those morphological and topological measurements have not in the past been applied to joint biomarkers.

A quantitative measure, which can be one or more of curvature, topology and shape, can be made of each joint biomarker.

### BRIEF DESCRIPTION OF THE DRAWINGS

A preferred embodiment of the present invention will be set forth in detail with reference to the drawings, in which:

FIG. 1 shows a flow chart of an overview of the process of the preferred embodiment;

FIG. 2 shows a flow chart of a segmentation process used in the process of FIG. 1;

FIG. 3 shows a process of tracking a segmented image in multiple images taken over time;

FIG. 4 shows a block diagram of a system on which the process of FIGS. 1-3 can be implemented; and

FIG. 5 shows an image of a biomarker formed in accordance with the preferred embodiment.

### DETAILED DESCRIPTION OF THE PREFERRED EMBODIMENT

A preferred embodiment of the present invention will now be set forth with reference to the drawings.

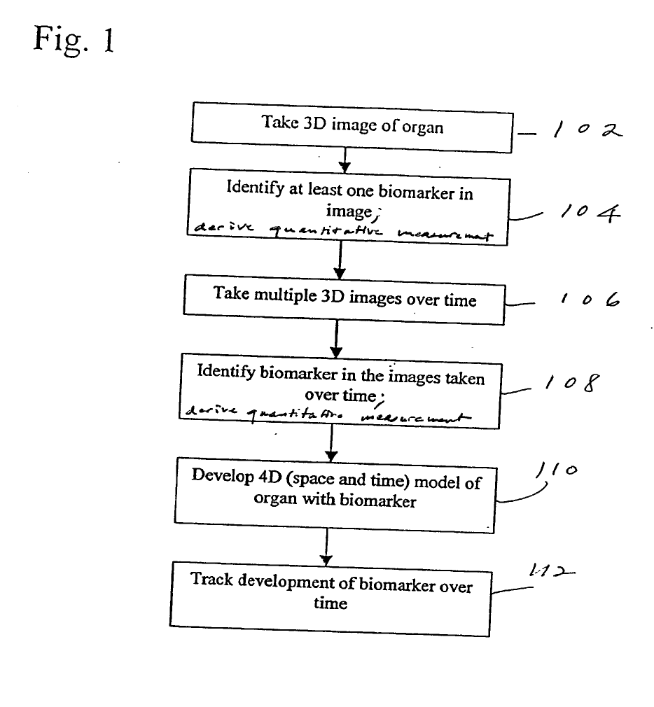

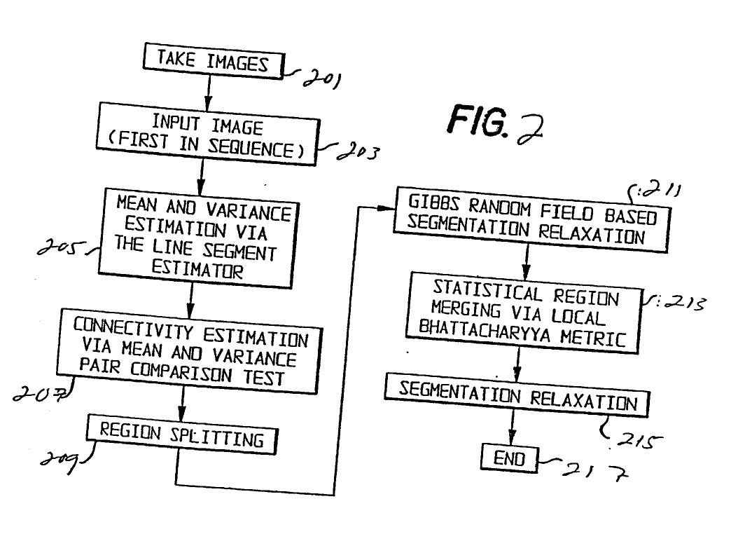

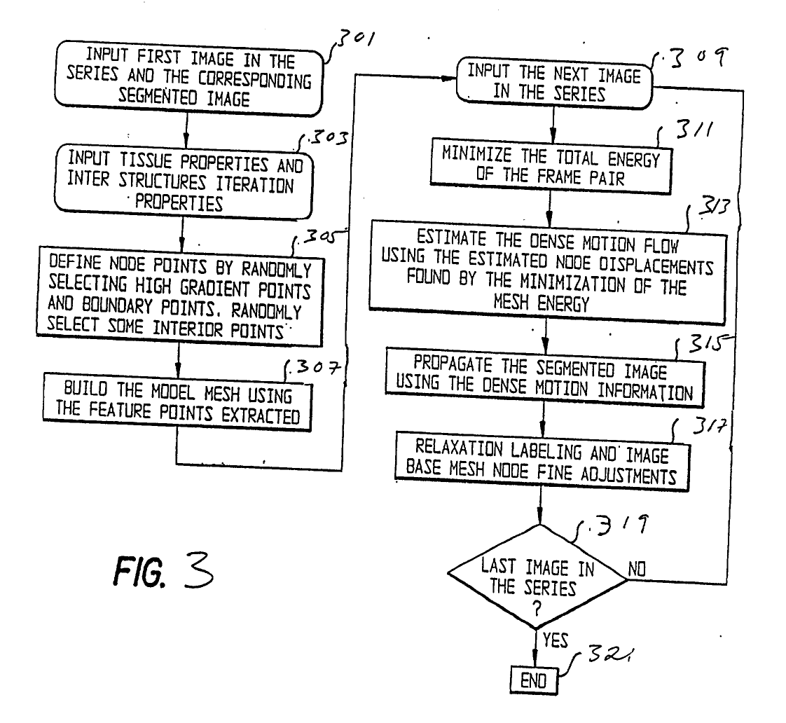

FIG. 1 shows an overview of the process of identifying biomarkers and their trends over time. In step 102, a three-dimensional image of the organ is taken. In step 104, at least one biomarker is identified in the image; the technique for doing so will be explained with reference to FIG. 2. Also in step 104, at least one quantitative measurement is made of the biomarker. In step 106, multiple three-dimensional images of the same region of the organ are taken over time. In some cases, step 106 may be completed before step 104; the order of those steps is a matter of convenience. In step 108, the same biomarker or biomarkers and their quantitative measurements are identified in the images taken over time; the technique for doing so will be explained with reference to FIG. 3. The identification of the biomarkers in the multiple image allows the development in step 110 of a model of the organ in four dimensions, namely, three dimensions of space and one of time. From that model, the development of the biomarker or biomarkers can be tracked over time in step 112.

The preferred method for extracting the biomarkers is with statistical based reasoning as defined in Parker et al (U.S. Pat. No. 6,169,817), whose disclosure is hereby incorporated by reference in its entirety into the present disclosure. From raw image data obtained through magnetic resonance imaging or the like, an object is reconstructed and visualized in four dimensions (both space and time) by first dividing the first image in the sequence of images into regions through statistical estimation of the mean value and variance of the image data and joining of picture elements (voxels) that are sufficiently similar and then extrapolating the regions to the remainder of the images by using known motion characteristics of components of the image (e.g., spring constants of muscles and tendons) to estimate the rigid and deformational motion of each region from image to image. The object and its regions can be rendered and interacted with in a four-dimensional (4D) virtual reality environment, the four dimensions being three spatial dimensions and time.

The segmentation will be explained with reference to FIG. 2. First, at step 201, the images in the sequence are taken, as by an MRI. Raw image data are thus obtained. Then, at step 203, the raw data of the first image in the sequence are input into a computing device. Next, for each voxel, the local mean value and region variance of the image data are estimated at step 205. The connectivity among the voxels is estimated at step 207 by a comparison of the mean values and variances estimated at step 205 to form regions. Once the connectivity is estimated, it is determined which regions need to be split, and those regions are split, at step 209. The accuracy of those regions can be improved still more through the segmentation relaxation of step 211. Then, it is determined which regions need to be merged, and those regions are merged, at step 213. Again, segmentation relaxation is performed, at step 215. Thus, the raw image data are converted into a segmented image, which is the end result at step 217. Further details of any of those processes can be found in the above-cited Parker et al patent.

The creation of a 4D model (in three dimensions of space and one of time) will be described with reference to FIG. 3. A motion tracking and estimation algorithm provides the information needed to pass the segmented image from one frame to another once the first image in the sequence and the completely segmented image derived therefrom as described above have been input at step 301. The presence of both the rigid and non-rigid components should ideally be taken into account in the estimation of the 3D motion. According to the present invention, the motion vector of each voxel is estimated after the registration of selected feature points in the image.

To take into consideration the movement of the many structures present in a joint, the approach of the present invention takes into account the local deformations of soft tissues by using a priori knowledge of the material properties of the different structures found in the image segmentation. Such knowledge is input in an appropriate database form at step 303. Also, different strategies can be applied to the motion of the rigid structures and to that of the soft tissues. Once the selected points have been registered, the motion vector of every voxel in the image is computed by interpolating the motion vectors of the selected points. Once the motion vector of each voxel has been estimated, the segmentation of the next image in the sequence is just the propagation of the segmentation of the former image. That technique is repeated until every image in the sequence has been analyzed. Note that the definition of time and the order of a sequence can be reversed for convenience in the analysis.

Finite-element models (FEM) are known for the analysis of images and for time-evolution analysis. The present invention follows a similar approach and recovers the point correspondence by minimizing the total energy of a mesh of masses and springs that models the physical properties of the anatomy. In the present invention, the mesh is not constrained by a single structure in the image, but instead is free to model the whole volumetric image, in which topological properties are supplied by the first segmented image and the physical properties are supplied by the a priori properties and the first segmented image. The motion estimation approach is an FEM-based point correspondence recovery algorithm between two consecutive images in the sequence. Each node in the mesh is an automatically selected feature point of the image sought to be tracked, and the spring stiffness is computed from the first segmented image and a priori knowledge of the human anatomy and typical biomechanical properties for muscle, bone and the like.

Many deformable models assume that a vector force field that drives spring-attached point masses can be extracted from the image. Most such models use that approach to build semi-automatic feature extraction algorithms. The present invention employs a similar approach and assumes that the image sampled at t=n is a set of three dynamic scalar fields:

Φ(x,t)={gₙ(x),|∇gₙ(x)|,∇²gₙ(x)},

namely, the gray-scale image value, the magnitude of the gradient of the image value, and the Laplacian of the image value. Accordingly, a change in Φ(x, t) causes a quadratic change in the scalar field energy U_{Φ}(x)∝(ΔΦ(x))². Furthermore, the structures underlying the image are assumed to be modeled as a mesh of spring-attached point masses in a state of equilibrium with those scalar fields. Although equilibrium assumes that there is an external force field, the shape of the force field is not important. The distribution of the point masses is assumed to change in time, and the total energy change in a time period Δt after time t=n is given by

$$Δ {U}_{n} ( Δ x ) = {\sum }_{∀ X ∈ {g}_{n}} [ {( α ( {g}_{n} ( x ) - {g}_{n + 1} ( x + Δ x ) ) )}^{2} + {( β ( \vert ∇ {g}_{n} ( x ) \vert - \vert ∇ {g}_{n + 1} ( x + Δ x ) \vert ) )}^{2} + {( γ ( {∇}^{2} {g}_{n} ( x ) + {∇}^{2} {g}_{n + 1} ( x + Δ x ) ) )}^{2} + \frac{1}{2} ηΔ {X}^{T} K Δ X ]$$

where α, β, and γ are weights for the contribution of every individual field change, η weighs the gain in the strain energy, K is the FEM stiffness matrix, and ΔX is the FEM node displacement matrix. Analysis of that equation shows that any change in the image fields or in the mesh point distribution increases the system total energy. Therefore, the point correspondence from gₙ to gₙ₊₁ is given by the mesh configuration whose total energy variation is a minimum. Accordingly, the point correspondence is given by

\hat{X}=X+Δ\hat{X}

where

Δ\hat{X}=min_{ΔX}ΔUₙ(ΔX).

In that notation, minₚ q is the value of p that minimizes q.

While the equations set forth above could conceivably be used to estimate the motion (point correspondence) of every voxel in the image, the number of voxels, which is typically over one million, and the complex nature of the equations make global minimization difficult. To simplify the problem, a coarse FEM mesh is constructed with selected points from the image at step 305. The energy minimization gives the point correspondence of the selected points.

The selection of such points is not trivial. First, for practical purposes, the number of points has to be very small, typically≅10⁴; care must be taken that the selected points describe the whole image motion. Second, region boundaries are important features because boundary tracking is enough for accurate region motion description. Third, at region boundaries, the magnitude of the gradient is high, and the Laplacian is at a zero crossing point, making region boundaries easy features to track. Accordingly, segmented boundary points are selected in the construction of the FEM.

Although the boundary points represent a small subset of the image points, there are still too many boundary points for practical purposes. In order to reduce the number of points, constrained random sampling of the boundary points is used for the point extraction step. The constraint consists of avoiding the selection of a point too close to the points already selected. That constraint allows a more uniform selection of the points across the boundaries. Finally, to reduce the motion estimation error at points internal to each region, a few more points of the image are randomly selected using the same distance constraint. Experimental results show that between 5,000 and 10,000 points are enough to estimate and describe the motion of a typical volumetric image of 256×256×34 voxels. Of the selected points, 75% are arbitrarily chosen as boundary points, while the remaining 25% are interior points. Of course, other percentages can be used where appropriate.

Once a set of points to track is selected, the next step is to construct an FEM mesh for those points at step 307. The mesh constrains the kind of motion allowed by coding the material properties and the interaction properties for each region. The first step is to find, for every nodal point, the neighboring nodal point. Those skilled in the art will appreciate that the operation of finding the neighboring nodal point corresponds to building the Voronoi diagram of the mesh. Its dual, the Delaunay triangulation, represents the best possible tetrahedral finite element for a given nodal configuration. The Voronoi diagram is constructed by a dilation approach. Under that approach, each nodal point in the discrete volume is dilated. Such dilation achieves two purposes. First, it is tested when one dilated point contacts another, so that neighboring points can be identified. Second, every voxel can be associated with a point of the mesh.

Once every point xᵢ has been associated with a neighboring point xⱼ, the two points are considered to be attached by a spring having spring constant k_{i,j}^{l,m}, where l and m identify the materials. The spring constant is defined by the material interaction properties of the connected points; those material interaction properties are predefined by the user in accordance with known properties of the materials. If the connected points belong to the same region, the spring constant reduces to k_{i,j}^{l,m} and is derived from the elastic properties of the material in the region. If the connected points belong to different regions, the spring constant is derived from the average interaction force between the materials at the boundary. If the object being imaged is a human shoulder, the spring constant can be derived from a table such as the following:

| | Humeral head | Muscle | Tendon | Cartilage |
|---|---|---|---|---|
| Humeral head | 10⁴ | 0.15 | 0.7 | 0.01 |
| Muscle | 0.15 | 0.1 | 0.7 | 0.6 |
| Tendon | 0.7 | 0.7 | 10 | 0.01 |
| Cartilage | 0.01 | 0.6 | 0.01 | 10² |

In theory, the interaction must be defined between any two adjacent regions. In practice, however, it is an acceptable approximation to define the interaction only between major anatomical components in the image and to leave the rest as arbitrary constants. In such an approximation, the error introduced is not significant compared with other errors introduced in the assumptions set forth above.

Spring constants can be assigned automatically, as the approximate size and image intensity for the bones are usually known a priori. Segmented-image regions matching the a priori expectations are assigned to the relatively rigid elastic constants for bone. Soft tissues are assigned relatively soft elastic constants.

Once the mesh has been set up, the next image in the sequence is input at step 309, and the energy between the two successive images in the sequence is minimized at step 311. The problem of minimizing the energy U can be split into two separate problems: minimizing the energy associated with rigid motion and minimizing that associated with deformable motion. While both energies use the same energy function, they rely on different strategies.

The rigid motion estimation relies on the fact that the contribution of rigid motion to the mesh deformation energy (ΔX^{T}KΔX)/2 is very close to zero. The segmentation and the a priori knowledge of the anatomy indicate which points belong to a rigid body. If such points are selected for every individual rigid region, the rigid motion energy minimization is accomplished by finding, for each rigid region Rᵢ, the rigid motion rotation Rᵢ and the translation Tᵢ that minimize that region's own energy:

$$Δ {X}_{rigid} = {min}_{Δ x} {U}_{rigid} = {\sum }_{∀ l ∈ rigid} ( Δ \hat{X} = {min}_{Δ {x}_{i}} {U}_{n} ( Δ {X}_{i} ) )$$

where ΔXᵢ=Rᵢ·Xᵢ+TᵢXᵢ and Δ\hat{x}ᵢ is the optimum displacement matrix for the points that belong to the rigid region Rᵢ. That minimization problem has only six degrees of freedom for each rigid region: three in the rotation matrix and three in the translation matrix. Therefore, the twelve components (nine rotational and three translational) can be found via a six-dimensional steepest-descent technique if the difference between any two images in the sequence is small enough.

Once the rigid motion parameters have been found, the deformational motion is estimated through minimization of the total system energy U. That minimization cannot be simplified as much as the minimization of the rigid energy, and without further considerations, the number of degrees of freedom in a 3D deformable object is three times the number of node points in the entire mesh. The nature of the problem allows the use of a simple gradient descent technique for each node in the mesh. From the potential and kinetic energies, the Lagrangian (or kinetic potential, defined in physics as the kinetic energy minus the potential energy) of the system can be used to derive the Euler-Lagrange equations for every node of the system where the driving local force is just the gradient of the energy field. For every node in the mesh, the local energy is given by

$${U}_{{X}_{i} , n} ( Δ x ) = {( α ( {g}_{n} ( {x}_{i} + Δ x ) - {g}_{n + 1} ( {x}_{i} ) ) )}^{2} + {( β ( \vert ∇ {g}_{n} ( {x}_{i} + Δ x ) \vert - \vert ∇ {g}_{n + 1} ( {x}_{i} ) \vert ) )}^{2} + {( γ ( {∇}^{2} {g}_{n} ( {x}_{i} + Δ x ) + {∇}^{2} {g}_{n + 1} ( {x}_{i} ) ) )}^{2} + \frac{1}{2} η {\sum }_{{x}_{i} ∈ {G}_{m} ( {X}_{i} )} {( {k}_{i , j}^{l , m} ( {x}_{j} - {x}_{i} - Δ x ) )}^{2}$$

where Gₘ represents a neighborhood in the Voronoi diagram.

Thus, for every node, there is a problem in three degrees of freedom whose minimization is performed using a simple gradient descent technique that iteratively reduces the local node energy. The local node gradient descent equation is

xᵢ(n+1)=xᵢ(n)−vΔU₍ₓ_{i(n),n)}(Δx)

where the gradient of the mesh energy is analytically computable, the gradient of the field energy is numerically estimated from the image at two different resolutions, x(n+1) is the next node position, and v is a weighting factor for the gradient contribution.

At every step in the minimization, the process for each node takes into account the neighboring nodes' former displacement. The process is repeated until the total energy reaches a local minimum, which for small deformations is close to or equal to the global minimum. The displacement vector thus found represents the estimated motion at the node points.

Once the minimization process just described yields the sampled displacement field ΔX, that displacement field is used to estimate the dense motion field needed to track the segmentation from one image in the sequence to the next (step 313). The dense motion is estimated by weighting the contribution of every neighbor mode in the mesh. A constant velocity model is assumed, and the estimated velocity of a voxel x at a time t is v(x, t)=Δx(t)/Δt. The dense motion field is estimated by

$$v ( x , t ) = \frac{c ( x )}{Δ t} {\sum }_{∀ Δ {x}_{j} ∈ {G}_{m} ( {x}_{i} )} \frac{{k}^{l , m} Δ {x}_{j}}{\vert x - {x}_{j} \vert} where$$

$$c ( x ) = {[ {\sum }_{∀ Δ {x}_{j} ∈ {G}_{m} ( {x}_{i} )} \frac{{k}^{l , m}}{\vert x - {x}_{j} \vert} ]}^{- 1}$$

k^{l,m} is the spring constant or stiffness between the materials l and m associated with the voxels x and xⱼ, Δt is the time interval between successive images in the sequence, |x−xⱼ| is the simple Euclidean distance between the voxels, and the interpolation is performed using the neighbor nodes of the closest node to the voxel x. That interpolation weights the contribution of every neighbor node by its material property k_{i,j}^{l,m}; thus, the estimated voxel motion is similar for every homogeneous region, even at the boundary of that region.

Then, at step 315, the next image in the sequence is filled with the segmentation data. That means that the regions determined in one image are carried over into the next image. To do so, the velocity is estimated for every voxel in that next image. That is accomplished by a reverse mapping of the estimated motion, which is given by

$$v ( x , t + Δ t ) = \frac{1}{H} {\sum }_{∀ [ {x}_{j} + v ( {x}_{j} , t ) ] ∈ S ( x )} v ( {x}_{j} , t )$$

where H is the number of points that fall into the same voxel space S(x) in the next image. That mapping does not fill all the space at time t+Δt, but a simple interpolation between mapped neighbor voxels can be used to fill out that space. Once the velocity is estimated for every voxel in the next image, the segmentation of that image is simply

L(x,t+Δt)=L(x−v(x,t+Δt)Δt,t)

where L(x,t) and L(x,t+Δt) are the segmentation labels at the voxel x for the times t and t+Δt.

At step 317, the segmentation thus developed is adjusted through relaxation labeling, such as that done at steps 211 and 215, and fine adjustments are made to the mesh nodes in the image. Then, the next image is input at step 309, unless it is determined at step 319 that the last image in the sequence has been segmented, in which case the operation ends at step 321.

The operations described above can be implemented in a system such as that shown in the block diagram of FIG. 4. System 400 includes an input device 402 for input of the image data, the database of material properties, and the like. The information input through the input device 402 is received in the workstation 404, which has a storage device 406 such as a hard drive, a processing unit 408 for performing the processing disclosed above to provide the 4D data, and a graphics rendering engine 410 for preparing the 4D data for viewing, e.g., by surface rendering. An output device 412 can include a monitor for viewing the images rendered by the rendering engine 410, a further storage device such as a video recorder for recording the images, or both. Illustrative examples of the workstation 304 and the graphics rendering engine 410 are a Silicon Graphics Indigo workstation and an Irix Explorer 3D graphics engine.

Shape and topology of the identified biomarkers can be quantified by any suitable techniques known in analytical geometry. The preferred method for quantifying shape and topology is with the morphological and topological formulas as defined by the references cited above.

As one example of the quantitative measurement of new biomarkers, the knee of an adult human was scanned with a 1.5 Tesla MRI system, with an in-plane resolution of 0.3 mm and a slice thickness of 2.0 mm. The cartilage of the femur, tibia, and fibia were segmented using the statistical reasoning techniques of Parker et al (cited above). Characterization of the cartilage structures was obtained by applying morphological and topological measurements. One such measurement is the estimation of local surface curvature. Techniques for the determination of local surface curvature are well known in analytic geometry. For example, if S(x,y,z) is the surface of a structure with an outward normal <n> the mean curvature, a local quantity can be determined from the roots of a quadratic equation found in Struik (cited above), p. 83. The measurements provide a quantitative, reproducible, and very sensitive characterization of the cartilage, in a way which is not practical using conventional manual tracings of 2D image slices.

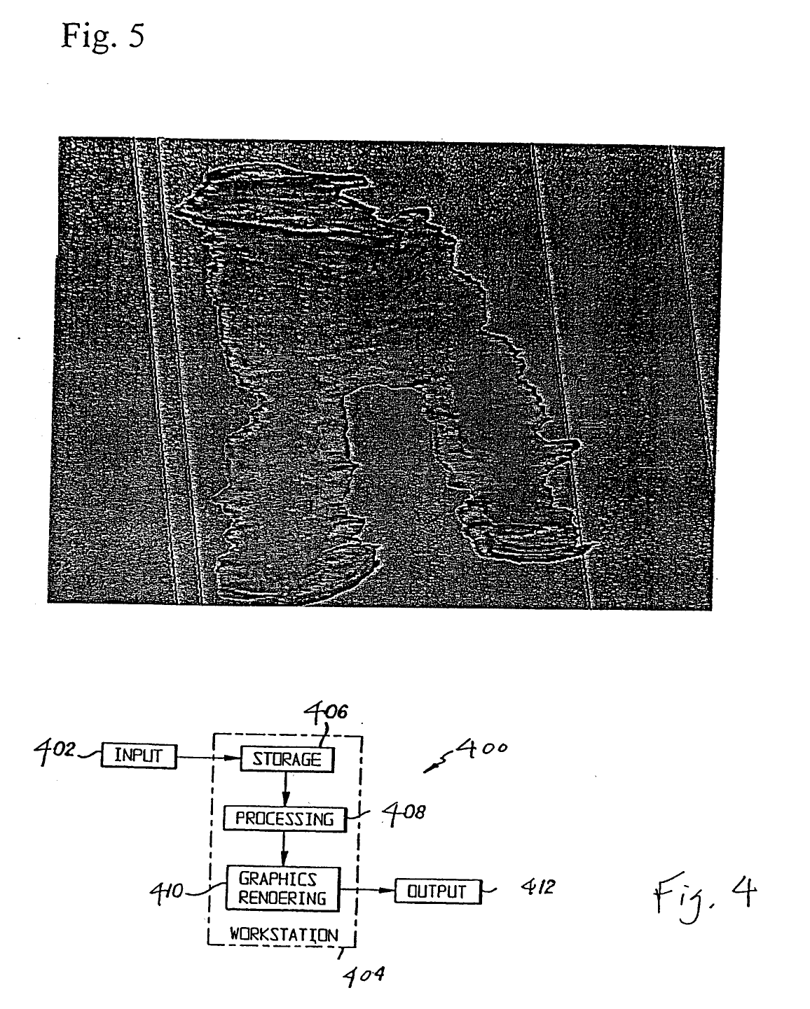

FIG. 5 provides a gray scale graph of the quantitative higher order measure of surface curvature, as a function of location within the surface of the cartilage. The view is from the upper femur, looking down towards the knee to the inner surface of the cartilage. Shades of dark-to-light indicate quantitative measurements of local curvature, a higher order measurement.

Those data are then analyzed over time as the individual is scanned at later intervals. There are two types of presentations of the time trends that are preferred. In one class, the repeated higher order measurements are as shown as in FIG. 5, with successive measurements overlaid in rapid sequence so as to form a movie. In the complementary representation, a trend plot is drawn giving the higher order measures as a function of time. For example, the mean and standard deviation (or range) of the local curvature can be plotted for a specific cartilage local area, as a function of time.

The accuracy of those measurements and their sensitivity to subtle changes in small substructures are highly dependent on the resolution of the imaging system. Unfortunately, most CT, MRI, and ultrasound systems have poor resolution in the out-of-plane, or “z” axis. While the in-plane resolution of those systems can commonly resolve objects that are just under one millimeter in separation, the out-of-plane (slice thickness) is commonly set at 1.5 mm or even greater. For assessing subtle changes and small defects using higher order structural measurements, it is desirable to have better than one millimeter resolution in all three orthogonal axes. That can be accomplished by fusion of a high resolution scan in the orthogonal, or out-of-plane direction, to create a high resolution voxel data set (Peña, J.-T., Totterman, S. M. S., Parker, K. J. “MRI Isotropic Resolution Reconstruction from Two Orthogonal Scans,” SPIE Medical Imaging, 2001, hereby incorporated by reference in its entirety into the present disclosure). In addition to the assessment of subtle defects in structures, that high-resolution voxel data set enables more accurate measurement of structures that are thin, curved, or tortuous.

In following the response of a person or animal to therapy, or to monitor the progression of disease, it is desirable to accurately and precisely monitor the trends in biomarkers over time. That is difficult to do in conventional practice since repeated scans must be reviewed independently and the biomarkers of interest must be traced or measured manually or semi-manually with each time interval representing a new and tedious process for repeating the measurements. It is highly advantageous to take a 4D approach, such as was defined in the above-cited patent to Parker et al, where a biomarker is identified with statistical reasoning, and the biomarker is tracked from scan to scan over time. That is, the initial segmentation of the biomarker of interest is passed on to the data sets from scans taken at later intervals. A search is done to track the biomarker boundaries from one scan to the next. The accuracy and precision and reproducibility of that approach is superior to that of performing manual or semi-manual measurements on images with no automatic tracking or passing of boundary information from one scan interval to subsequent scans.

The quantitative assessment of the new biomarkers listed above provides an objective measurement of the state of the joints, particularly in the progression of joint disease. It is also very useful to obtain accurate measurements of those biomarkers over time, particularly to judge the degree of response to a new therapy, or to assess the trends with increasing age. Manual and semi-manual tracings of conventional biomarkers (such as the simple thickness or volume of the cartilage) have a high inherent variability, so as successive scans are traced the variability can hide subtle trends. That means that only gross changes, sometimes over very long time periods, can be verified in conventional methods. The inventors have discovered that by extracting the biomarker using statistical tests, and by treating the biomarker over time as a 4D object, with an automatic passing of boundaries from one time interval to the next, provides a highly accurate and reproducible segmentation from which trends over time can be detected. Thus, the combination of selected biomarkers that themselves capture subtle pathologies, with a 4D approach to increase accuracy and reliability over time, creates sensitivity that has not been previously obtainable.

While a preferred embodiment of the invention has been set forth above, those skilled in the art who have reviewed the present disclosure will readily appreciate that other embodiments can be realized within the scope of the present invention. For example, any suitable imaging technology can be used. Therefore, the present invention should be construed as limited only by the appended claims.

We claim:

1. A method for assessing a joint of a patient, the method comprising:
(a) taking at least one three-dimensional image of the joint;
(b) identifying at least one biomarker in the at least one three-dimensional image;
(c) deriving at least one quantitative measurement of the at least one biomarkers; and
(d) storing an identification of the at least one biomarker and the at least one quantitative measurement in a storage medium.

2. The method of claim 1, wherein step (d) comprises storing the at least one three-dimensional image in the storage medium.

3. The method of claim 1, wherein step (b) comprises statistical segmentation of the at least one three-dimensional image to identify the at least one biomarker.

4. The method of claim 1, wherein the at least one three-dimensional image comprises a plurality of three-dimensional images of the joint taken over time.

5. The method of claim 4, wherein step (b) comprises statistical segmentation of a three-dimensional image selected from the plurality of three-dimensional images to identify the at least one biomarker.

6. The method of claim 5, wherein step (b) further comprises motion tracking and estimation to identify the at least one biomarker in the plurality of three-dimensional images in accordance with the at least one biomarker identified in the selected three-dimensional image.

7. The method of claim 6, wherein the plurality of three-dimensional images and the at least one biomarker identified in the plurality of three-dimensional images are used to form a model of the joint and the at least one biomarker in three dimensions of space and one dimension of time.

8. The method of claim 7, wherein the biomarker is tracked over time in the model.

9. The method of claim 1, wherein a resolution in all three dimensions of the at least one three-dimensional image is finer than 1 mm.

10. The method of claim 9, wherein the at least one quantitative measurement comprises a higher order quantitative measurement.

11. The method of claim 10, wherein the higher order quantitative measurement comprises at least one of curvature, topology and shape.

12. The method of claim 1, wherein the at least one biomarker is selected from the group consisting of:
shape of a subchondral bone plate;
layers of cartilage and their relative size;
signal intensity distribution within the cartilage layers;
contact area between articulating cartilage surfaces;
surface topology of a cartilage shape;
intensity of bone marrow edema;
separation distances between bones;
meniscus shape;
meniscus surface area;
meniscus contact area with cartilage;
cartilage structural characteristics;
cartilage surface characteristics;
meniscus structural characteristics;
meniscus surface characteristics;
pannus structural characteristics;
joint fluid characteristics;
osteophyte characteristics;
bone characteristics;
lytic lesion characteristics;
prosthesis contact characteristics;
prosthesis wear;
joint spacing characteristics;
tibia medial cartilage volume;
tibia lateral cartilage volume;
femur cartilage volume;
patella cartilage volume;
tibia medial cartilage curvature;
tibia lateral cartilage curvature;
femur cartilage curvature;
patella cartilage curvature;
cartilage bending energy;
subchondral bone plate curvature;
subchondral bone plate bending energy;
meniscus volume;
osteophyte volume;
cartilage T2 lesion volumes;
bone marrow edema volume and number;
synovial fluid volume;
synovial thickening;
subchondrial bone cyst volume;
kinematic tibial translation;
kinematic tibial rotation;
kinematic tibial valcus;
distance between vertebral bodies;
degree of subsidence of cage;
degree of lordosis by angle measurement;
degree of off-set between vertebral bodies;
femoral bone characteristics; and
patella characteristics.

13. The method of claim 1, wherein step (a) is performed through magnetic resonance imaging.

14. A system for assessing a joint of a patient, the system comprising:
(a) an input device for receiving at least one three-dimensional image of the joint;
(b) a processor, in communication with the input device, for receiving the at least one three-dimensional image of the joint, identifying at least one biomarker in the at least one three-dimensional image and deriving at least one quantitative measurement of the at least one biomarker;
(c) storage, in communication with the processor, for storing the at least one three-dimensional image, an identification of the at least one biomarker and the at least one quantitative measurement; and
(d) an output device for displaying the at least one three-dimensional image, the identification of the at least one biomarker and the at least one quantitative measurement.

15. The system of claim 14, wherein the storage also stores the at least one three-dimensional image.

16. The system of claim 14, wherein the processor identifies the at least one biomarker through statistical segmentation of the at least one three-dimensional image.

17. The system of claim 14, wherein the at least one three-dimensional image comprises a plurality of three-dimensional images of the joint taken over time.

18. The system of claim 17, wherein the processor identifies the at least one biomarkers through statistical segmentation of a three-dimensional image selected from the plurality of three-dimensional images.

19. The system of claim 18, wherein the processor uses motion tracking and estimation to identify the at least one biomarker in the plurality of three-dimensional images in accordance with the at least one biomarker identified in the selected three-dimensional image.

20. The system of claim 19, wherein the plurality of three-dimensional images and the at least one biomarker identified in the plurality of three-dimensional images are used to form a model of the joint and the at least one biomarker in three dimensions of space and one dimension of time.

21. The system of claim 14, wherein a resolution in all three dimensions of the at least one three-dimensional image is finer than 1 mm.

22. The system of claim 14, wherein the at least one quantitative measurement comprises a higher order quantitative measurement.

23. The system of claim 22, wherein the higher order quantitative measurement comprises at least one of curvature, topology and shape.

24. The system of claim 14, wherein the at least one biomarker is selected from the group consisting of:
shape of a subchondral bone plate;
layers of cartilage and their relative size;
signal intensity distribution within the cartilage layers;
contact area between articulating cartilage surfaces;
surface topology of a cartilage shape;
intensity of bone marrow edema;
separation distances between bones;
meniscus shape;
meniscus surface area;
meniscus contact area with cartilage;
cartilage structural characteristics;
cartilage surface characteristics;
meniscus structural characteristics;
meniscus surface characteristics;
pannus structural characteristics;
joint fluid characteristics;
osteophyte characteristics;
bone characteristics;
lytic lesion characteristics;
prosthesis contact characteristics;
prosthesis wear;
joint spacing characteristics;
tibia medial cartilage volume;
tibia lateral cartilage volume;
femur cartilage volume;
patella cartilage volume;
tibia medial cartilage curvature;
tibia lateral cartilage curvature;
femur cartilage curvature;
patella cartilage curvature;
cartilage bending energy;
subchondral bone plate curvature;
subchondral bone plate bending energy;
meniscus volume;
osteophyte volume;
cartilage T2 lesion volumes;
bone marrow edema volume and number;
synovial fluid volume;
synovial thickening;
subchondrial bone cyst volume;
kinematic tibial translation;
kinematic tibial rotation;
kinematic tibial valcus;
distance between vertebral bodies;
degree of subsidence of cage;
degree of lordosis by angle measurement;
degree of off-set between vertebral bodies;
femoral bone characteristics; and
patella characteristics.
